(19) United States
(12) Patent Application Publication
(10) Pub. No.: US 2024/0358448 A1
(43) Pub. Date: Oct. 31, 2024

(54) MULTI-UNIT DEVICE FOR ROBOTIC MANIPULATION OF ELONGATE SURGICAL TOOLS

(71) Applicant: Microbot Medical Ltd., Yokneam Illit (IL)

(72) Inventors: Idan BOADER, Karmiel (IL); Simon SHARON, Zichron Yaacov (IL); Eran COHEN, Kiryat-Tivon (IL); Eyal MORAG, Tel Aviv (IL)

(73) Assignee: Microbot Medical Ltd., Yokneam Illit (IL)

(21) Appl. No.: 18/292,956

(22) PCT Filed: Jul. 13, 2022
(86) PCT No.: PCT/IL2022/050756

§ 371 Date: Jan. 29, 2024

Related U.S. Application Data
(60) Provisional application No. 63/226,800, filed on Jul. 29, 2021.

(51) Int. Cl. A61B 34/30; A61M 25/00; A61M 25/09

(52) U.S. Cl. CPC A61B 34/30; A61M 25/0021; A61M 25/09; A61B 2034/301; A61M 2025/0042

(57) ABSTRACT

A robotic device for manipulation of an arrangement of a plurality of elongate surgical tools, comprising: a first unit and a second unit, each unit configured to receive at least one elongate surgical tool of the arrangement, where an elongate surgical tool received by the first unit is different from an elongate surgical tool received by the second unit; each unit comprising a driving assembly and motors configured for actuation of the driving assembly to move the at least one elongate surgical tool; and a coupler attached to the first unit and to the second unit, the coupler configured so that movement of the two or more portions relative to each other allows movement of at least one of the first and second units relative to the other unit, while in each of the first and second units the elongate surgical tool remains in operable engagement with the driving assembly.

### RELATED APPLICATION/S

This application claims the benefit of priority of U.S. Provisional Patent Application No. 63/226,800 filed on 29 Jul. 2021, the contents of which are incorporated herein by reference in their entirety.

### FIELD AND BACKGROUND OF THE INVENTION

The present invention, in some embodiments thereof, relates to robotic manipulation of elongate surgical tools and, more particularly, but not exclusively, to a multi-unit device configured to receive and drive movement of a pre-assembled arrangement of tools.

### SUMMARY OF THE INVENTION

According to an aspect of some embodiments there is provided a robotic device for manipulation of an arrangement of a plurality of elongate surgical tools, comprising:
a first motive unit and a second motive unit, each motive unit configured to receive at least one elongate surgical tool of the arrangement, wherein an elongate surgical tool received by the first motive unit is different from an elongate surgical tool received by the second motive unit; each motive unit comprising a driving assembly and one or more motors configured for actuation of the driving assembly to move the at least one elongate surgical tool; and
a coupler attached to the first motive unit and to the second motive unit, the coupler comprising two or more portions connected to each other; the coupler configured so that movement of the two or more portions relative to each other allows movement of at least one of the first and second motive units relative to the other motive unit, while in each of the first and second motive units the elongate surgical tool remains in operable engagement with the driving assembly.

In some embodiments, at least the first motive unit is attached to the coupler by a reversible attachment which provides for replacing at least the first motive unit as a whole.

In some embodiments, the coupler is configured to hold the first motive unit relative to the second motive unit at more than one spatial configuration.

In some embodiments, the more than one spatial configuration includes a vertical arrangement of the first motive unit relative to the second motive unit, such that long axes of the driving assemblies of the units are parallel to each other; and an inclined arrangement in which a long axis of the driving assembly of the first motive unit extends at an angle relative to a long axis of the driving assembly of the second motive unit.

In some embodiments, in each of the first and second motive units, no barrier exists between the driving assembly and the elongate surgical tool received within the motive unit.

In some embodiments, in each of the motive units, no barrier exists between the driving assembly and the one or more motors.

In some embodiments, one of the at least two portions of the coupler is attached to the first motive unit, and the other of the at least two portions of the coupler is attached to the second motive unit.

In some embodiments, movement of the two or more portions of the coupler is spring actuated.

In some embodiments, the elongate surgical tool received within the first motive unit comprises a guidewire, and the elongate surgical tool received within the second motive unit comprises a microcatheter, the arrangement comprising the guidewire at least partially inserted into a lumen of the microcatheter.

In some embodiments, the driving assembly of the first motive unit is positioned to contact the guidewire when the guidewire is received within the first motive unit and to one or both of move the guidewire linearly and roll the guidewire; and wherein the driving assembly of the second motive unit is positioned to contact the micro catheter when the microcatheter is received within the second motive unit and to move the microcatheter linearly.

In some embodiments, the second motive unit comprises an attachment to a guiding catheter, the microcatheter and guidewire arrangement being at least partially inserted into a lumen of the guiding catheter.

In some embodiments, the second motive unit is slidably mounted on a platform, and sliding movement of the second motive unit relative to the platform linearly moves the attached guiding catheter along with the inserted microcatheter and guidewire.

In some embodiments, the first motive unit is shaped as a barrel, and the coupler holds the first motive unit vertically above the second motive unit.

In some embodiments, the two or more portions of the coupler comprise a lever portion and a vertical rail which are connected to each other at a joint.

In some embodiments, the arrangement of a plurality of elongate surgical tools manipulated by the device comprises a telescopic arrangement, and the elongate surgical tool received by the first motive unit is at least partially introduced into a lumen of the elongate surgical tool received by the second motive unit.

In some embodiments, the elongate surgical tool received by the first motive unit comprise a guidewire and the elongate surgical tool received by the second motive unit comprises a microcatheter.

According to an aspect of some embodiments there is provided a device for manipulation of one or more elongate surgical tools, comprising:
a stage defining a path along which at least one elongate surgical tool is received, the path shaped for aligning the elongate surgical tool when the elongate tool is received therein; and
an actuation portion shaped and sized to be selectively mounted onto the stage, the actuation portion comprising a driving assembly and one or more motors configured for actuation of the driving assembly; wherein mounting of the actuation portion onto the stage places the driving assembly in contact with the at least one elongate surgical tool such that the driving assembly is operable to move the at least one elongate surgical tool.

In some embodiments, the actuation portion is formed as a cover of the stage to be placed on top of the stage.

In some embodiments, the actuation portion is shaped to enclose the path.

In some embodiments, the device comprises at least one interlock which when locked restrains relative movement between the actuation portion and the stage.

In some embodiments, housings of the stage and the actuation portion are similar at least in axial length.

In some embodiments, when the actuation portion is mounted on the stage, a long axis of the driving assembly is parallel to a long axis of the path, the axes extending along a similar vertical plane.

In some embodiments, the driving assembly comprises a plurality of pairs of opposing wheels arrayed adjacent each other, wherein the long axis of the driving assembly extends along spaces defined between opposing wheels of each of the plurality pairs.

According to an aspect of some embodiments there is provided a method of setting up a robotic device for manipulation of one or more elongate surgical tools, the robotic device comprising at least: a stage; and an actuation portion including one or more motors for actuating a driving assembly which moves at least one of the elongate surgical tools, the method comprising:
placing at least one surgical tool on the stage such that the elongate surgical tool is aligned along the stage by a designated path;
coupling the actuation portion to the stage, wherein the coupling places the driving assembly in alignment and in contact with the elongate surgical tool.

In some embodiments, the actuation portion is configured as a cover of the stage, and coupling comprises closing the actuation portion over the stage.

In some embodiments, the driving assembly comprises a plurality of wheel pairs, and the coupling positions the wheel pairs such that opposing wheels of each pair are placed on either side of the elongate surgical tool.

In some embodiments, the one or more elongate surgical tools include at least one of: a guidewire, a microcatheter, a guiding catheter.

In some embodiments, the method further comprises, prior to the placing, assembling the one or more elongate surgical tools together as a telescopic arrangement; and placing the telescopic arrangement in the designated path.

In some embodiments, the method further comprises, prior to the placing, introducing the one or more elongate surgical tools into the patient body.

In some embodiments, the method further comprises, prior to the placing and following the introducing, approximating the stage to an entry point of the one or more elongate surgical tools into the patient body.

In some embodiments, the method further comprises, prior to the placing, positioning an adaptor onto the at least one elongate surgical tool, and wherein the placing comprises inserting the adaptor into a designated recess on the stage.

According to an aspect of some embodiments there is provided a method of effectively increasing a useable length of an elongate surgical tool manipulated by a robotic device which includes at least first and second units, the elongate surgical tool extending from an attachment with the first unit, between the first and second units, and then through a designated path of the second unit, the method comprising:
identifying a need to increase a useable length of the elongate surgical tool;
while maintaining the elongate surgical tool in attachment with the first unit and inside the designated path of the second unit, approximating an attachment of the elongate surgical tool to the first unit to an entry point of the elongate surgical tool into the designated path of the second tool, such that an additional segment of the elongate surgical tool is made available for use.

In some embodiments, the method further comprises advancing the elongate surgical tool through the designated path and further into a patient body.

In some embodiments, approximating comprises changing a spatial configuration of the first unit relative to the second unit via a coupler connected to the first and second units.

In some embodiments, changing comprises moving the first unit from a vertical position relative to the second unit to an inclined or horizontal position relative to the second unit.

In some embodiments, moving is spring actuated.

In some embodiments, the method comprises controlling the approximating remotely using a remote-controller.

In some embodiments, identifying comprises one or more of: identifying in response to a signal received from a controller of the robotic device, identifying in response to user input, identifying in response to an indication obtained by one or more sensors of the robotic device.

According to an aspect of some embodiments there is provided a robotic device for manipulation of an elongate surgical tool insertable into a patient body, the device comprising:
an elongate path for receipt of the elongate surgical tool;
one or more motors;
a driving assembly configured to move the elongate surgical tool when the tool is received within the path, the driving assembly actuated by the one or more motors;
wherein the elongate path includes at least one axially offset portion which sets a controlled buckling location for the elongate surgical tool if the elongate surgical tool encounters resistance when being advanced within the patient body.

In some embodiments, the axially offset portion comprises a curved portion extending away from a long axis of the elongate path.

According to an aspect of some embodiments there is provided a robotic device for manipulation of an elongate surgical tool insertable into a patient body, the device comprising:
an elongate path for receipt of the elongate surgical tool, the elongate path including;
one or more motors;
a driving assembly configured to move the elongate surgical tool when the tool is received within the path, the driving assembly actuated by the one or more motors; and
one or more sensors positioned at or adjacent the elongate path and configured for detecting one or both of contact of the elongate surgical tool with one or more walls of the path and force applied by the elongate surgical tool on the one or more walls of the path.

In some embodiments, the elongate path comprises an axially offset portion and the one or more sensors are positioned at or adjacent the axially offset portion.

In some embodiments, the axially offset portion comprises a curved portion.

In some embodiments, the one or more sensors comprise a pressure sensor.

In some embodiments, the robotic device comprises a controller configured to receive indications from the one or more sensors and to actuate the one or more motors to move the elongate surgical tool by the driving assembly when the one or more sensors detect contact and/or force applied by the elongate surgical tool.

In some embodiments, the controller is configured to retract the elongate surgical tool proximally to a position in which resistance encountered by a distal end of the elongate surgical tool is avoided.

In some embodiments, the controller is configured to generate an alert informing a user that the elongate surgical tool is buckled or bent.

According to an aspect of some embodiments there is provided a motive unit for manipulation of a guidewire, comprising:
a housing including:
a guidewire pre-loaded onto a designated path inside the housing;
one or more motors;
a driving assembly configured to move the guidewire, the driving assembly actuated by the one or more motors;
wherein the housing defines an external attachment to a coupler shaped and configured to hold the motive unit relative to at least a second motive unit.

In some embodiments, the driving assembly comprises a plurality of wheel pairs positioned in contact with the guidewire, wherein rotation of the wheels moves the guidewire linearly and wherein rotation of the driving assembly as a single unit rolls the guidewire as the guidewire is held between opposing wheels of the plurality of wheel pairs.

In some embodiments, the housing is shaped as a barrel and wherein a diameter of the barrel is sized according to a radius of rotation of the driving assembly as a single unit.

In some embodiments, the housing defines an attachment for a handle of the guidewire.

In some embodiments, there is provided a robotic device comprising:
a motive unit for example as described herein;
a second motive unit; and
a coupler comprising two or more rigid portions that are adjustable in position relative to each other, a first portion configured to attach to the first motive unit and a second portion configured to attach to the second motive unit, wherein movement of the rigid portions relative to each other provides for selectively positioning the first motive unit relative to the second motive unit.

In some embodiments, the second motive unit is configured for manipulation of a microcatheter, and the guidewire is at least partially inserted into a lumen of the microcatheter.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method of preparing an arrangement of elongate surgical tools and loading the arrangement onto a robotic device, according to some embodiments;

FIGS. 2A-B are schematic block diagrams of a robotic device for manipulation of one or more elongate surgical tools, according to some embodiments;

FIGS. 3A-B are different views of a robotic device loaded with elongate surgical tools, according to some embodiments;

FIG. 4 is a partial exploded view of the robotic device of FIGS. 3A-B, showing movement driving assemblies for the surgical elongate tools received in the robotic device, according to some embodiments;

FIG. 5 is a perspective view of an alignment stage of a base of the robotic device of FIGS. 3A-B, on which an arrangement of elongate surgical tools is mounted;

FIGS. 6A-B are partial views of a part of the alignment stage showing tool alignment and fixation to the alignment stage via an adaptor coupled to the tool, according to some embodiments;

FIG. 7 is a flowchart of a method for changing a spatial configuration of the robotic device, according to some embodiments;

FIGS. 8A-C are side views of an exemplary embodiment of the robotic device in a vertical setup and in an inclined setup, according to some embodiments;

FIG. 9 is a flowchart of a method for detection and/or control of buckling and/or bending of an elongate surgical tool at least partially received within the robotic device, according to some embodiments;

FIGS. 10A-B schematically illustrate a non-linear path for an elongate surgical tool where sensing of buckling and/or bending of the tool is performed, according to some embodiments;

FIG. 11 is a cross section of a portion of the robotic device including a non-linear path for an elongate surgical tool, according to some embodiments;

FIGS. 12A-D schematically illustrate an attachment for a robotic device for manipulation of one or more elongate surgical tools, according to some embodiments.

### DESCRIPTION OF SPECIFIC EMBODIMENTS OF THE INVENTION

The present invention, in some embodiments thereof, relates to robotic manipulation of elongate surgical tools and, more particularly, but not exclusively, to a multi-unit device configured to receive and drive movement of a pre-assembled arrangement of tools.

A broad aspect of some embodiments relates to manipulation of an arrangement of elongate surgical tools, for example, a telescopic arrangement of tools, by a device comprising multiple motive units where each motive unit is configured to engage and optionally drive movement of at least one of the tools of the arrangement.

In some embodiments, an assembled arrangement of tools, including, for example, a guidewire at least partially received within a lumen of a microcatheter, and a guiding catheter in which the guidewire-microcatheter arrangement is at least partially received, is loaded onto the robotic device. In some embodiments, the device is shaped and configured so that placing of the arrangement aligns the tools relative to the device so that one or more driving assemblies of the device are then positioned to operably engage the tools to move them (e.g. to move the tool linearly and/or roll the tool).

In some embodiments, loading of the arrangement of tools onto the device is performed after one or more of the tools are inserted into the patient body, for example, a guiding catheter is introduced into the patient body via an entry point, the arrangement of tools is assembled, and only then loaded onto the device. A potential advantage of loading of the tool(s) onto the device only simultaneously and/or after tool(s) were at least partially introduced into the patient body may include that the device can be selectively positioned relative to the patient body, for example, positioned in close proximity to the entry point, once established.

In some embodiments, multiple motive units of the device include one or more units which engage tool(s) that are optionally replaced or changed during the surgical procedure, for example, a guidewire; while one or more other units engage tool(s) that are optionally used throughout the procedure, and are not changed or replaced, for example, a microcatheter and/or a guiding catheter. In some embodiments, the one or more units engaging tool(s) that are changed are interchangeable as a whole, for example, a unit which receives and drives movement of a guidewire is optionally replaced along with the guidewire. In some embodiments, a guidewire is pre-mounted onto a unit for example so that pre-loading of the guidewire onto the unit is not required, and the unit (inclusive of the pre-mounted guidewire) can be assembled as is onto the system (e.g. attached to the coupler and/or directly to other units).

An aspect of some embodiments relates to alignment of at least one elongate surgical tool relative to the device which manipulates its movement, and specifically relative to a driving assembly of the device which engages the tool to move it.

In some embodiments, a motive unit of the device comprises a stage which is shaped to define a path for receipt of the tool (or, in some embodiments, a telescopic arrangement of tools), such that when the tool is placed on the path, the tool is aligned by the path relative to the stage portion (e.g. relative to a length and/or a width of the stage top surface). In some embodiments, the stage does not include components which are configured to move the tool (e.g. motors, driving assemblies) but rather functions as a cast on which the tool is mounted and which shapes the tool to have it fit within a designated path. In some embodiments, the stage includes no powering and/or electrical components. In some embodiments, the stage is relatively compact and lightweight, potentially facilitating placement of the stage at a desired location relative to the patient and/or relative to other surgical room equipment (e.g. surgical bed, imaging modalities, etc.) In some embodiments, prior to loading of the assembly of elongate surgical tools onto stage, the stage itself can be brought in proximity to the ready assembly of tools, or vice versa.

In some embodiments, the device comprises an actuation portion which is selectively positioned in attachment with the stage, for example, the actuation portion is formed as a cover that is closed over the stage. The actuation portion includes a driving assembly (and optionally, one or more motors which actuate the driving assembly) so that when the actuation portion is attached to the stage, the driving assembly is placed in operable engagement with the tool that is mounted onto the stage. In an example, the tool is received within an elongate slot defined on a surface of the stage. Upon closing of the actuation portion on top of the stage, a driving assembly of the actuation portion is aligned relative to the designated path, for example so that a long axis of the driving assembly is parallel with the path and is in close proximity to the path, allowing the driving assembly to contact the tool.

In some embodiments, the actuation portion rests on the stage, for example, being placed on top of the stage (vertically above the stage) such that gravitational forces facilitate positioning of the actuation portion.

In some embodiments, the actuation portion is placed on the stage only after the stage was selectively located (e.g. by a physician, surgeon), so that motive components of the actuation portion (such as motors, powering and the like) are brought into an operable position relative to the surgical tool only after the tool was aligned by the stage.

In some embodiments, the stage defines one or more designated recesses in which adaptor(s) of a tool (or of an arrangement of tools) are received. Optionally, by placing an adaptor (or a part of the adaptor, such as a Y-connector of a tool) in its designated recess, an axial alignment and/or rotational orientation of the tool relative to its designated path may be obtained.

An aspect of some embodiments relates to adjusting a spatial configuration of multiple units of a robotic device which manipulates one or more elongate surgical tools.

In some embodiments, two or more units of device are connected via a coupler, for example, a frame which includes at least two portions (e.g. a lever portion and a vertical rail) that are moveable relative to each other, for example via a joint coupling. In some embodiments, a first portion of the coupler is attached to a first unit, and a second portion of the coupler is attached to a second unit. When at least one of the portions is moved relative to the other, the portion repositions the unit that is attached to it relative to the other unit. In an example, the first unit is moved from a vertical arrangement in which it is held, for example, above the second unit, to an inclined or horizontal arrangement relative to the second unit.

In some embodiments, the change in spatial position of the units changes a relative position of driving assemblies of the units. For example, each of the units comprises a driving assembly including a plurality of wheel pairs arranged adjacent each other, where a long axis of the assembly is defined by the spaces between opposing wheels of each pair. When changing a spatial configuration of the device units, a relative position of the long axes of the driving assemblies is also changed, for example from being parallel (in a vertical alignment of the units) to being linearly aligned (in a horizontal alignment of the units) or being arranged at an angle relative to each other (in an inclined setup of the units).

In some embodiments, the change in spatial configuration is performed while the tool(s) are respectively held by their units, without the need to remove the tools before the change and without losing hold of the tool during or after the change. Optionally, the units operable to continue moving the tools even during the change itself.

In some embodiments, the coupler is adjustable so that it can hold the units at a plurality of different spatial positions relative to each other. Optionally, one or more positions are spring-biased (for example, the coupler transforms from one position to another in response to spring actuation). In some embodiments, the coupler is configured to lock at a certain positon to restrict relative movement of the units at that position.

An aspect of some embodiments relates to increasing a usable length of a tool manipulated by a robotic device. In some embodiments, an increase in useable length is achieved by the change in spatial configuration of the device units. In an example, the units are moved in closer proximity to each other (or more specifically, an attachment point and/or an entry location of the tool to a first unit is placed closer to an attachment point and/or an entry location of the tool to a second unit). In some embodiments, the change in spatial configuration allows for a shorter segment of the tool to extend between the two units (for example as compared to the length of a segment that extended between the units prior to the change in spatial configuration). By that, additional tool length can be used (manipulated) by the robotic device, for example, additional tool length may be advanced distally through a designated path of a unit. A potential advantage of increasing a usable length of a tool may include that in situations in which, for example, a distal end of a tool inserted into the body needs to be advanced further, a spatial configuration of the device units can be changed (optionally, automatically upon identifying the need for more usable tool length), and the tool can be advanced further inside the body.

An aspect of some embodiments relates to control of a buckling or bending location of an elongate surgical tool manipulated by the device. In some embodiments, control of a location in which the tool is bent or buckled, such as an axial location along the length of the tool and/or a location relative to a designated path of the device in which the tool passes, is achieved by leading the tool through at least one axially-offset position, for example curved segment of the designated path. In some embodiments, the axially offset position includes a position that is located away from a linear axis of the path. In some embodiments, when the tool encounters resistance (such as by a distal tip of the tool facing an obstruction, optionally inside the patient body), the tool may bend or buckle; since the tool segment that passes along the axially-offset position of the path is likely to bend first (for example as it is more “prone” to continue bending relative to straightened tool segments), the axially-offset position would set at least an initial buckling or bending of the tool to that location.

An aspect of some embodiments relates to determining a condition (e.g. buckling, bending) of an elongate surgical tool manipulated by the device by detecting contact and/or force applied by the tool onto wall(s) of the path, optionally at an axially offset (e.g. a curved) segment of the path. In some embodiments, one or more sensors (such as force (e.g. pressure) sensors, contact sensors, proximity sensors) are located at or adjacent the curved segment of the path and are configured to detect whether a tool passing through the curved segment is encountering resistance, for example at its distal end, which causes the tool to bend or buckle.

As referred to herein, the term “proximal” may refer to device and/or tool portions and/or directions that are further away from the patient body, for example, farther from an entry point to the patient body; the term “distal” may refer to device and/or tool portions and/or directions that are closer to the patient body, for example, farther towards and optionally deeper into an entry point to the patient body and/or towards a target location within the patient body.

As referred to herein, a “motive unit” or a “unit” may include a device portion which is optionally separable from one or more other device portions, In some embodiments, the unit is a tool-receiving unit, for example including a designated path for receipt of an elongate surgical tool and/or an attachment (optionally, external to the unit) to an elongate surgical tool. In some embodiments, a unit includes a housing (e.g. box shaped, barrel shaped, and/or otherwise shaped housing) where walls of the housing define between them an inner volume. In some embodiments, the inner volume accommodates the designated path (e.g. a slot, a recess, or an arbitrary path) for receipt of a segment of a surgical tool. In some embodiments, the inner volume accommodates one or more motors. In some embodiments, the inner volume accommodates one or more driving assemblies, where a driving assembly includes, for example, a plurality of wheels configured to engage the tool received within the path and to move the tool. In some embodiments, the unit includes one or more attachments to a surgical tool (optionally to a tool different than the one received within the path), where an attachment is optionally located externally to the housing (e.g. formed as a protrusion of the housing).

In some embodiments, a unit is comprised of two or more portions, where, for example, a first portion that is configured to receive the tool (e.g. in a designated slot or recess) and a second portion which is configured to actuate movement of the tool when placed in contact with the first portion. In some embodiments, the first portion defines a “resting surface” for the tool and includes no motors and no powering components, and optionally no electrical connections.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Exemplary Preparation and Loading of Elongate Surgical Tools onto a Robotic Device

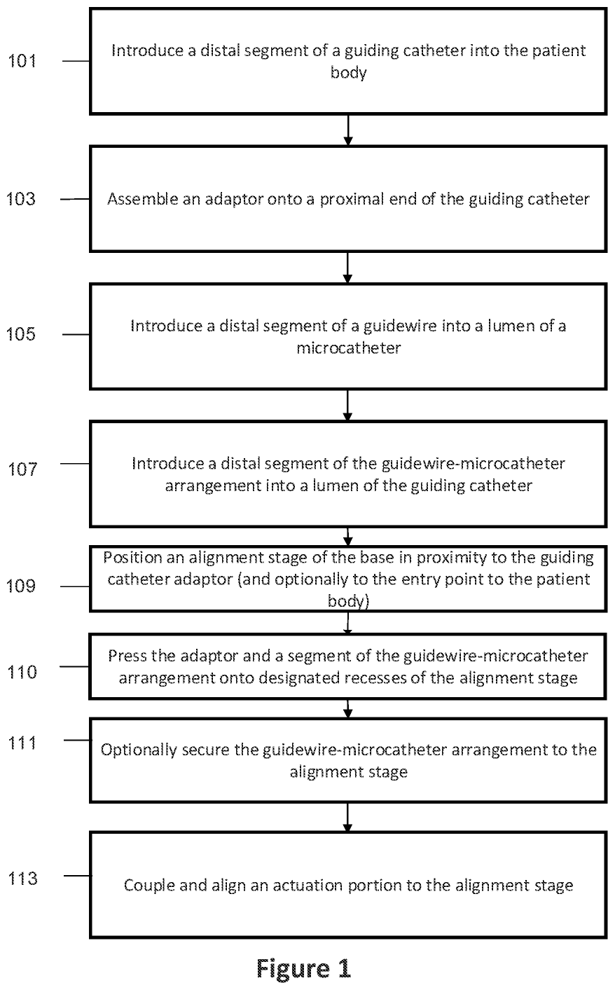

Referring now to the drawings, FIG. 1 is a flowchart of a method of preparing an arrangement of elongate surgical tools and loading the arrangement onto a robotic device, according to some embodiments.

In some embodiments, an arrangement of elongate surgical tools, for example including a guidewire, a microcatheter and a guiding catheter is prepared (e.g. by a user such as a physician, surgeon, nurse, technician and/or other clinical personnel) prior to loading of at least a portion of the arrangement onto the robotic device which manipulates movement of one or more tools of the arrangement.

In some embodiments, at least a portion of the arrangement of tools is introduced into the body of the patient, optionally prior to loading of the arrangement onto the robotic device. In an example, a distal segment of a guiding catheter is introduced into the patient body (101), for example via an entry point to the body. In some embodiments, depending on the location of the target tissue (for example, the heart, a peripheral blood vessel in the lower extremities, brain, liver, and the like) and the purpose of the procedure, the entry point may be selected from, but not limited to, at the patient's groin (i.e., the femoral artery), arm (i.e., the radial artery) or neck (i.e., the jugular vein). In some embodiments, the tool(s) are introduced into a blood vessel lumen.

In some embodiments, an adaptor (which optionally includes a connector to one or more other tools and/or devices and/or which allows injection of materials into a lumen of the device, for example, a Y-connector) is assembled onto a proximal end portion of the guiding catheter (103). In some embodiments, the adaptor further includes one or more transmission elements such as gears which when operably contacted with one or more other transmission elements and/or directly with a motor, are configured to move (e.g. rotate) the tool on which the adaptor is received.

In some embodiments, a user then takes a guidewire (optionally once removed from sterile package) and introduces the guidewire into a lumen of a microcatheter (which optionally was similarly removed from a sterile package), (105).

In some embodiments, at least a distal segment of the guidewire-microcatheter arrangement is inserted, optionally via a proximal end of the guiding catheter, into the lumen of the guiding catheter (107).

In some embodiments, once the user has completed setting up the assembly of elongate surgical tool, the user positions an alignment stage of the robotic device in proximity to the assembly of tools, and optionally, in proximity to the entry point to the patient body (109). Additionally or alternatively, the alignment stage of the device is pre-positioned at such location (e.g. placed on top or adjacent the patient bed when setting up the surgery room). In some embodiments, the device comprises a platform on which the alignment stage is mounted, and the platform (optionally along with the alignment stage) is placed in proximity to the entry point to the body. In some embodiments, the platform and mounting stage are selectively located with respect to the patient body after at least some of the tools of the assembly (optionally, all tools) were at least partially introduced into the patient body.

In some embodiments, the platform is mounted on an adjustable arm (or generally an adjustable rigid fixture) which is configured to hold the platform relative to the surgical bed and/or the patient. In some embodiments, one end of the arm is attached, for example, to the surgical bed, while the other end engages the platform.

In some embodiments, a user presses a segment of the guidewire-microcatheter arrangement and the adaptor onto designated recesses of the alignment stage (110). In some embodiments, the designated recess for the segment of the guidewire-microcatheter arrangement is an elongate recess extending, for example, along a long axis of the alignment stage, and by placing the arrangement into the recess, the segment of the arrangement is optionally straightened to extend linearly along the stage. Optionally, the segment is located by the recess with respect to the alignment stage (for example, centered relative to a width of the stage).

In some embodiments, the guiding catheter is situated such that its proximal end is located at or adjacent a face of the alignment stage which is facing towards the entry point to the patient body. In some embodiments, the guiding catheter adaptor is placed in its designated recess by linearly moving the adaptor and/or laterally moving the adaptor and/or by rotating the adaptor (without inducing rotation of the guiding catheter, about the long axis of the guiding catheter, until the adaptor is at least partially received within its designated recess.

In some embodiments, a user optionally secures the guidewire-microcatheter arrangement to the alignment stage (111), for example by closing a bridging element over a gap defined by the recess, while the segment is contained therein. Optionally, the bridging element is fixed to the alignment stage via a plurality of snap-fit connectors.

In some embodiments, once the arrangement of the tools is at least partially received within the alignment stage and optionally secured to it, a user couples and aligns an actuation portion of the device to the alignment stage (113).

In some embodiments, the actuation portion comprises one or more motors and a driving assembly actuated by the one or more motors. In some embodiments, the driving assembly is configured to drive linear and/or rotational movement of tool(s) of the arrangement, for example by a plurality of wheels configured and positioned to contact the tool to move it. In some embodiments, a plurality of wheels of a driving assembly configured inside the actuation portion contact the segment of the guidewire-microcatheter arrangement that was received within the designated recess of the alignment stage, and are configured to move at least the microcatheter linearly (e.g. advance or retract the microcatheter).

In some embodiments, the actuation portion is formed as a cover of the alignment portion which be selectively closed onto the alignment stage. When the cover is closed, the driving assembly is brought into operable contact with the segment of the arrangement of tools.

In some embodiments, the actuation portion does not necessarily form a topping cover of the alignment stage, but is formed, for example, as a side-engagement to the alignment stage, a bottom engagement to the alignment stage, and/or other arrangements suitable for enclosing and operably engaging a plurality of surgical tools mounted on the alignment stage (optionally, a telescopic assembly of the tools).

A potential advantage of a device setup and loading method for example as described herein may include that the user (e.g. physician, surgeon) assembles the telescopic arrangement of tools as optionally commonly performed and only then loads the arrangement onto the robotic device, while being able to position the device at a selected location relative to the body entry point. Commonly, the user would set up the assembly of tools on or near the patient bed and in proximity to the entry point into the body. Setting up the robotic device in proximity to the entry point into the patient body may be potentially advantageous in that: usable tool length may be effectively lengthened; a spatial and/or visual interference of the robotic device may be minimized; buckling of unengaged sections of tools may be reduced or avoided; a risk of obstruction by engaged sections of tools may be reduced or avoided.

### Multi-Unit Robotic Device

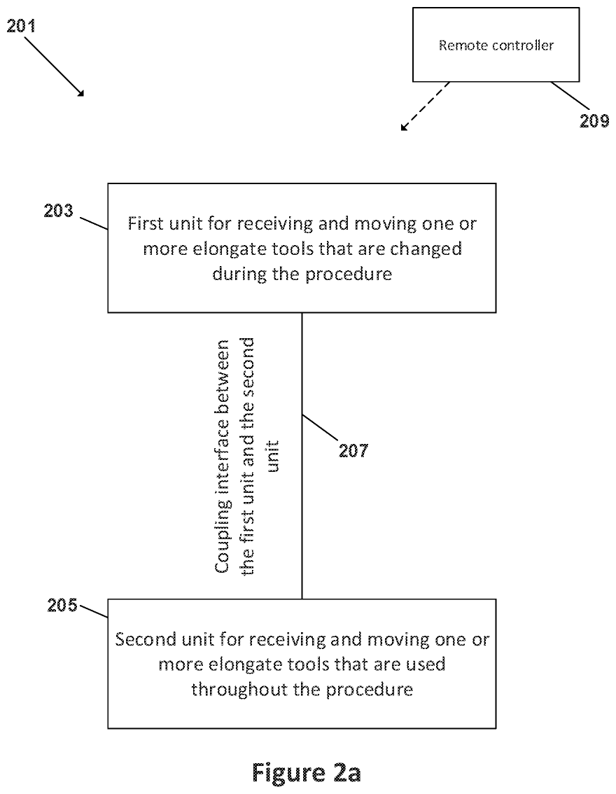

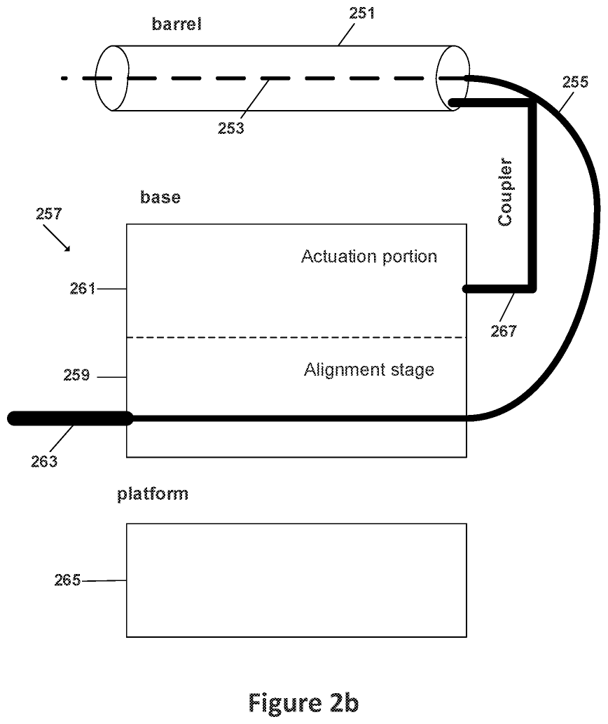

FIGS. 2A-B are schematic block diagrams of a robotic device for manipulation of one or more elongate surgical tools, according to some embodiments.

In some embodiments, as schematically shown by FIG. 2A, a robotic device 201 comprises a plurality of units, for example, 2 units, 3 units, 4 units, 5 units or larger or smaller number of units, where each unit is configured to receive and optionally to drive movement (e.g. linear movement and/or roll movement) of one or more elongate surgical tools.

In some embodiments, a unit includes a housing which defines an inner volume including a driving assembly (e.g. a set of wheels) which move the tool; and one or more motors for actuating the driving assembly. In some embodiments, the unit defines an elongate pathway in which at least a segment of the tool is received, and the driving assembly is positioned to come into operable contact with the tool segment when it is received within the pathway.

In some embodiments, device 201 comprises a first unit 203 which receives and moves one or more elongate surgical tools that are changed during a surgical procedure. In some embodiments, a tool is interchanged by another tool (e.g. a guidewire is switched to a detachable coil system, for example.). In some embodiments, a tool is replaced by a similar tool (e.g. a guidewire is replaced with a new guidewire). In some embodiments, the guidewire is retracted from the microcatheter to allow delivery of therapeutic agents, such as beads, coils, glue, etc.

In some embodiments, when changing a tool, the tool is removed from the first unit and the new tool is inserted. Alternatively, the first unit is replaced as a whole, optionally inclusive of the tool, by a new first unit in which the new tool is received and/or previously contained within. In some embodiments, the new tool is characterized by features different than the previous tool, for example, different stiffness levels, presence of a handle (such as in a steerable guidewire/catheter), existence of a delivery lumen and as such. In an example, replacing of the first unit as a whole (inclusive of the tool) may be advantageous when changing from a tool that does not include a proximal handle to a tool that does include a proximal handle (e.g. a steerable guidewire), and the new unit is optionally constructed to engage (and optionally move) that handle.

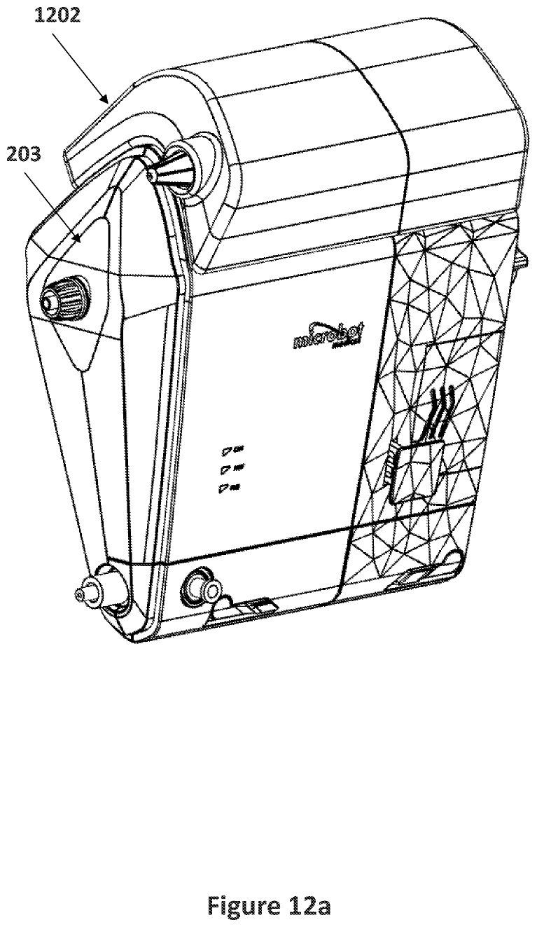

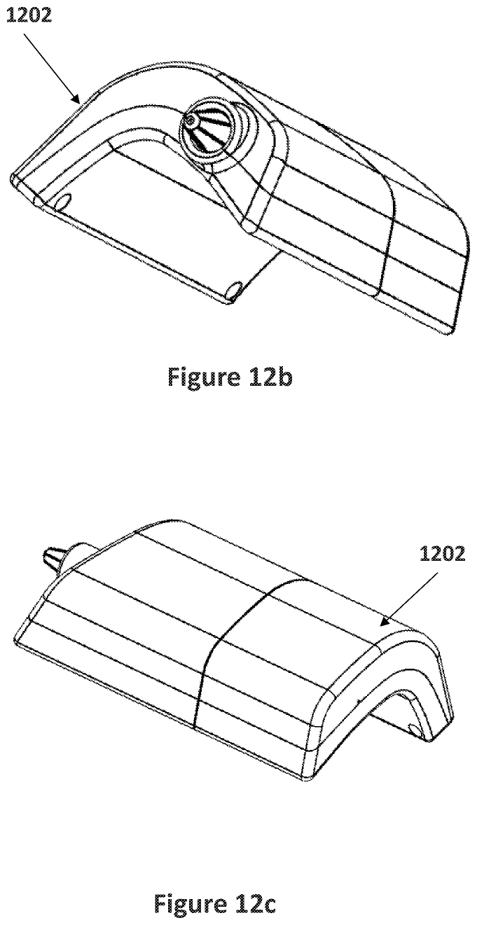

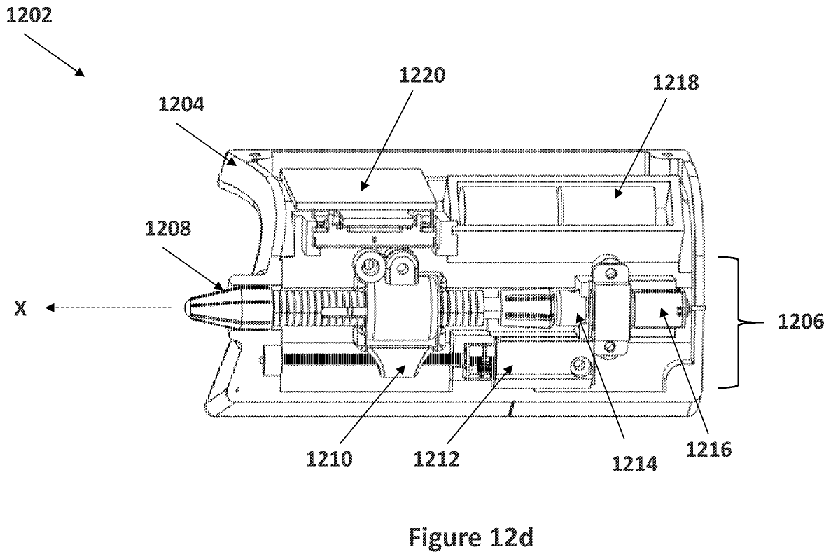

Referring now to FIGS. 12A-12D, showing schematic representations of an exemplary external attachment, according to some embodiments of the invention. In some embodiments, the first unit 203 includes an external attachment 1202 for a proximal handle of the tool, (e.g. a proximal handle of a steerable guidewire). In some embodiments, the attachment 1202 is configured on the external walls of the housing of the unit, as shown for example in FIG. 12A. In some embodiments, the attachment comprises a housing sized and shaped to fulfil one or more purposes: to house a proximal handle of the tool, to house the plurality of mechanisms configured to actuate the proximal handle of the tool and, optionally, to match the geometry of the first unit 203. FIG. 12A shows the attachment 1202 mounted on the first unit 203. As can be seen, the first unit 203 comprises an irregular shape, therefore, optionally, the housing of the attachment 1202 comprises a geometry that matches the geometry of the first unit 203. In some embodiments, a potential advantage of having matching geometries between the attachment 1202 and the first unit 203 is that is saves space when utilizing the attachment 1202, space that is not always available in the location where the medical procedure is performed. FIGS. 12B and 12C show perspectives views of the attachment 1202 having a housing comprising an irregular geometry matching the geometry of the first unit. For example, the top portion of the first unit 203 shown in FIG. 12a comprises a triangular shape. In some embodiments, the attachment comprises a geometry that matches the triangular shape of the top portion of the first unit 203, which is similar to an upside-down V, as shown for example in FIGS. 12A, 12B, 12C and 12D. In some embodiments, the internal components are arranged inside the irregular shape of the attachment, for example, one side of the attachment (one “leg” of the V shape) is used to accommodate the mechanical components of the attachment, while the other side (the other “leg” of the V shape) is used to accommodate the electronics, as shown for example in FIG. 12D. In some embodiments, the accommodation of the mechanical components on one side and the electrical components on the other side is designed and/or optimized in order to reduce the size of the attachment. Referring now to FIG. 12D, showing a schematic representation of exemplary components and/or mechanisms located inside the attachment 1202, according to some embodiments of the invention. In some embodiments, as mentioned above, the attachment 1202 comprises a housing 1204. In some embodiments, inside the housing 1204 there is a zone 1206 configured to receive the proximal handle of the tool 1208. In some embodiments, the attachment 1202 comprises a first actuation mechanism 1210, for example a worm screw mechanism, configured to actuate a slider located in the proximal handle of the tool 1208 (slider not shown). In some embodiments, the first actuation mechanism 1210 comprises a motor 1212 configured to actuate the first actuation mechanism 1210. In some embodiments, the attachment 1202 comprises a second actuation mechanism 1214, for example a rotational mechanism, configured to rotate the proximal handle of the tool 1208 along its own longitudinal axis “X”. In some embodiments, the second actuation mechanism 1214 comprises a motor 1216 configured to actuate the second actuation mechanism 1208. In some embodiments, the attachment 1202 comprises one or more power sources 1218, for example one or more batteries, configured to provide the necessary power to the attachment 1202. In some embodiments, an external power source is used to provide the necessary power to the attachment 1202. In some embodiments, the attachment 1202 comprises circuitry 1220 configured to control the overall actions of the attachment 1202. In some embodiments, the attachment 1202 comprises communication means, for example Wi-Fi, Bluetooth, etc., (not shown) in communication with the circuitry, and configured to connect the attachment 1202 with the rest of the system, which allows coordination and/or synchronization of actions between the attachment 1202 and the rest of the system. In some embodiments, the attachment is configured to actuate the tool by actuating the proximal handle of the tool. In some embodiments, the attachment is configured to rotate the tool by rotating the handle located within the attachment. Alternatively, in some embodiments, the attachment moves with the tool, for example, rotates along with the tool when the tool is rotated along with the driving assembly. It should be understood that other mechanisms could be used within the attachment 1202 to provide different actuations to the tool.

In some embodiments, for example if a tool with no handle was used and now a tool that includes a handle is to be used, the first unit is replaced by a different first unit which includes an attachment for the handle.

In some embodiments, device 201 comprises a second unit 205 which receives and moves one or more elongate tools that are used throughout the procedure, for example, without being changed or replaced. In an example, the second unit receives and moves a microcatheter, where optionally a portion of a guidewire received within the first unit 203 extends within an inner lumen of the microcatheter. In an example, the second unit interfaces with and moves a guiding catheter.

In some embodiments, a unit is configured to externally attach to one or more tools, for example externally to the unit housing. In an example, the first unit includes an attachment for connecting to a microcatheter (e.g. to a microcatheter proximal end).

In some embodiments, a coupling interface 207 exists between the first unit and the second unit. In some embodiments, the coupling interface is configured for one or more of: mechanically connecting the first and second units, electrically connecting the first and second units, transferring of actuation force (e.g. from one or more motors) from one unit to another, transferring of data (e.g. control signals, sensor readings, and/or other) from one unit to another.

In some embodiments, the coupling interface includes a coupler that extends between the two units and holds them together. In some embodiments, the coupler comprises a rigid fixture or frame which sets a relative position of the units with respect to each other and/or with respect to a platform on which the device is mounted. In some embodiments, the coupler holds the units along a substantially vertical axis, for example so that the first unit is held above the second unit. In some embodiments the coupler includes movable segments (e.g. a lever) which can be adjusted to set or modify a position of one unit relative to the other. In an example, the coupler is adjustable from a first position in which the first unit and the second unit are held along a vertical axis such that the first unit is held above the second unit; to a second position in which the first and second units are held along a horizontal axis (e.g. side by side) or the first unit is held at an angle (e.g. an angle smaller than 180 degrees) relative to the second unit.

In some embodiments, the coupler attaches to the unit(s) via a mechanical fixation, for example including pins, clips, screw-in attachments, snap fit attachments, and/or other fixation suitable for engaging the coupler and the unit and optionally for restraining relative movement of the unit, at least at its attachment to the coupler.

In some embodiments, the coupler is formed as a frame comprising portions (e.g. rods, beams or as such) shaped and sized to fit to the units' housings, for example, to extend along at least a portion of a wall of a unit housing.

In some embodiments, the coupling interface includes an electrical connection between the first and second units. Optionally, electrical power is supplied from the second unit to the first unit. In an example, electrical power is supplied via one or more slip rings which maintain electrical contact with the first unit at various rotational orientations of the first unit (for example if the first unit rotates when rolling the tool) and/or various positions of the first unit relative to the second. Additionally or alternatively, each unit is independently powered.

In some embodiments, the coupling interface is configured for data transfer between the units, for example, via wired or wireless communication. In some embodiments, the transferred data includes control signals for synchronizing movement of the tools by each of the units, for example so that a guidewire manipulated by the first unit moves along with a microcatheter (in which the guidewire is received) and which is manipulated by the second unit.

In some embodiments, the robotic device is controlled remotely, for example via a remote controller 209. In some embodiments, the remote controller includes a user interface for example configured as a joystick, console, computer or the like, from which a user controls operation of the device and/or receives input from the device (e.g. operational input, data obtained by device sensors, data obtained by encoders of the device and/or other). The remote controller may be used locally with the robotic device, for example when the user is present in the surgery room; or at a distance (remotely) from the device, for example when the user is in a different room.

In some embodiments, as shown for example in FIG. 2B, a first unit of the robotic device comprises a barrel 251 in which a proximal segment of a tool such as a guidewire 253 is received. In some embodiments, the barrel comprises an external attachment to another tool such as a proximal end of a microcatheter 255. In some embodiments, the guidewire extends from within the barrel and into the lumen of the microcatheter. In some embodiments, the barrel accommodates one or more motors which actuate a driving assembly for linearly moving and/or rolling the guidewire. In some embodiments, the barrel rotates as a single piece when the guidewire is rolled, for example, the external housing of the barrel rotates as well. Alternatively, at least a portion of an external housing of the barrel remains in place while inner components of the barrel (such as the driving assembly and/or one or more motors) rotate.

In some embodiments, a second unit of the robotic device comprises a base 257 which is comprised of at least: an alignment stage 259 on which the tool (e.g. microcatheter 255) is mounted and is aligned relative to the stage, for example arranged to lie along a long axis of the stage; and an actuation portion 261 which accommodates components for driving movement of the tool mounted on the alignment stage, such as one or more motors and a driving assembly.

In some embodiments, the actuation portion is formed as a cover of the alignment stage, which can be closed over the alignment stage to thereby place the driving assembly in operable contact with the tool mounted on the alignment stage. In an example, the alignment stage and the actuation portion are coupled by a hinge. In some embodiments, the actuation portion is fully detachable from the alignment stage and can be placed on the alignment stage (e.g. mounted onto the alignment stage, as a lid, cap or cover).

In some embodiments, the alignment stage includes an attachment for one or more elongate tools, for example, a guiding catheter 263. In some embodiments, the actuation portion comprises an additional driving assembly for moving the guiding catheter proximal end portion (e.g. rotating and/or linearly moving). Alternatively or additionally, the guiding catheter is provided with an adaptor including at least a portion of its driving assembly, for example, including a gear for interfacing with the motor found in the actuation portion and/or with transmission elements (e.g. gears) connected to the motor.

In some embodiments, in use, the guidewire is manipulated at the barrel; the microcatheter (in which the guidewire is received) is manipulated at the base; and optionally, the guiding catheter (in which the guidewire-microcatheter arrangement is received) is manipulated at the base. In some embodiments, one or more tools extend from an attachment point in one unit, optionally externally to the unit, and then into another unit. For example, the microcatheter extends from its attachment with the barrel (externally to the barrel), and then into its designated recess in the alignment stage of the base. Optionally, the microcatheter curves along a path passing between the barrel and the base, for example, forming a U-shaped curve.

In some embodiments, base 257 is seated on a platform 265. Optionally, the base is slideable relative to the platform. In some embodiments, linear movement of the guiding catheter is carried out by sliding of the base (optionally along with barrel 251) relative to the platform (e.g. back and forth along a long axis of the platform).

In some embodiments, device units are coupled via a coupler 267, for example, the base and the barrel are connected by the coupler. In some embodiments, a position of at least one of the barrel and the base relative to the other can be adjusted by a change in the coupler position and/or coupler structure.

In some embodiments, a device unit includes a housing that defines an inner volume in which the driving assembly and/or one or more motors are contained. In some embodiments, when the elongate surgical tool is received within the unit, the tool extends within the inner volume in a shared space with the driving assembly and/or one or more motors. In some embodiments, the tool, when received within its path, is in direct contact with the driving assembly.

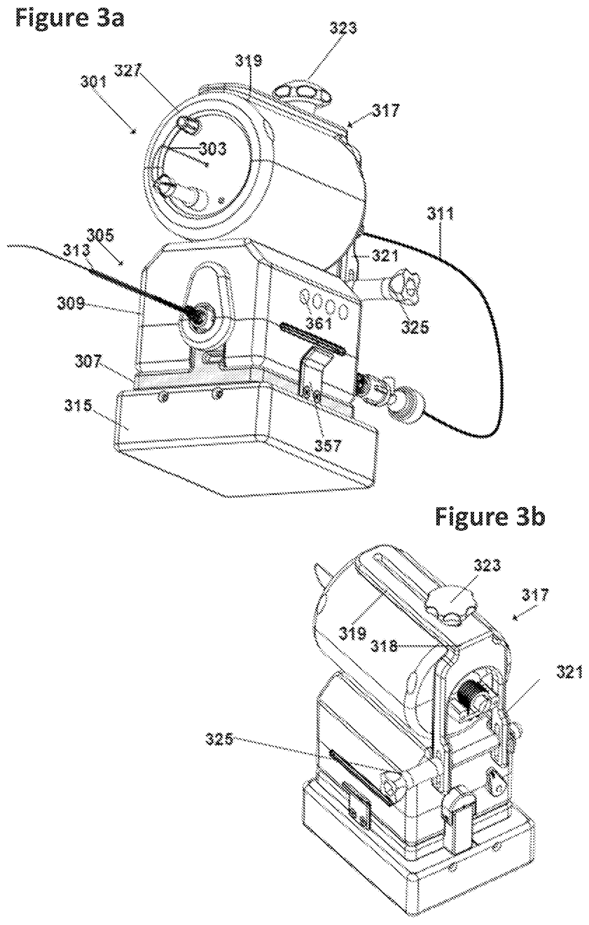

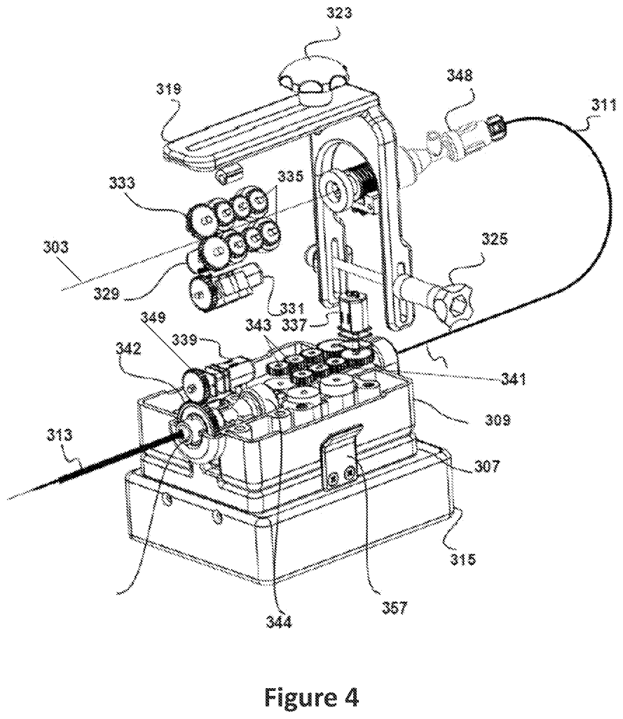

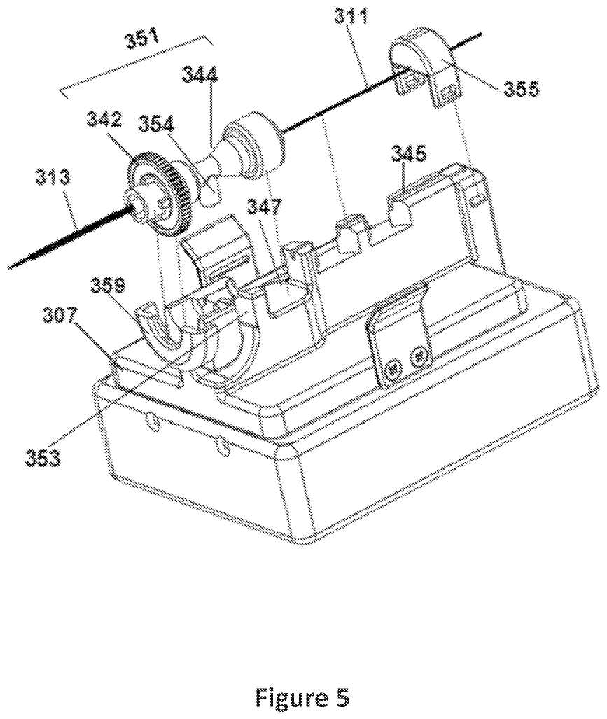

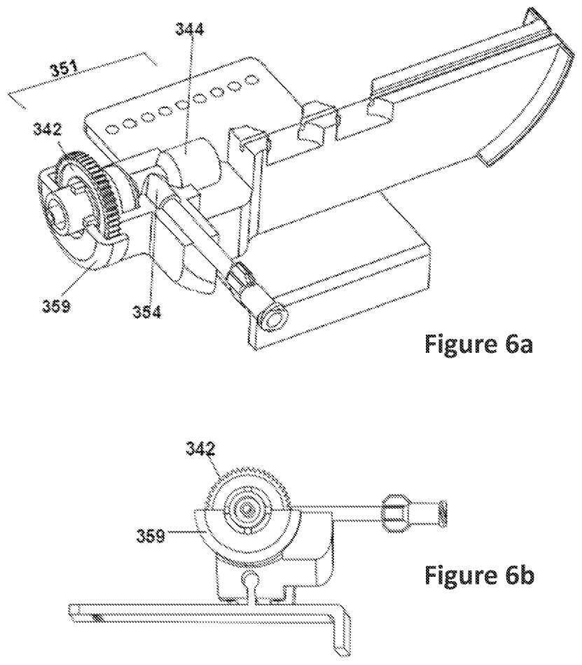

Reference is now made to: FIGS. 3A-B. showing an example of a robotic device loaded with elongate surgical tools, according to some embodiments; FIG. 4, showing a partial exploded view of the robotic device where the tool driving assemblies are visible; FIG. 5, which is a perspective views of an alignment stage of a base of the robotic device; and FIGS. 6A-B, which are partial views of a part an embodiment of the alignment stage showing tool alignment and fixation, according to some embodiments.

The exemplary robotic device, as shown for example in FIG. 3A, includes: a barrel 301 in which a guidewire 303 is received; a base 305 comprised of an alignment stage 307 and an actuation portion 309, where a microcatheter 311 is received; a guiding catheter 313 coupled to the base and extending from it; a platform 315 on which the base is moveably seated; and a coupler 317 (e.g. a frame) holding the barrel and the base relative to each other. A back view of the device is shown in FIG. 3B.

In the example shown, the barrel is stacked on top of the base, and optionally maintained in such alignment with the aid of the coupler 317. In some embodiments, the coupler comprises an L-shape structure including a lever portion 319 which extends, at least in part, along a length of the barrel, optionally on a top surface of the barrel; and a vertical rail 321 which is coupled to the lever and extends from the lever down towards the base, to which it is coupled. In some embodiments, the lever portion can be moved relative to the vertical rail, for example via a hinge joint 318 (see FIG. 3B) between them, so that an angle in which the lever portion extends relative to the vertical rail is adjustable. Alternatively, in some embodiments, the lever portion is fixed to the vertical rail and the two portions move together, for example when adjusting a position of the barrel relative to the base.

In some embodiments, the coupler comprises one or more adjustment interfaces such as knobs 323, 325 which, when rotated and/or pressed on, change a position of the lever portion relative to the vertical rail or vice versa, for example so as to change a relative position between the barrel and base.

In some embodiments, the coupler 317 is shaped and configured to allow moving the barrel vertically away or towards the base; holding the barrel at an angle relative to the base; setting an axial (linear) position of the barrel relative to the base; and/or otherwise allow relative positioning of the barrel and base.

Moving on to the barrel 301, in some embodiments, the barrel includes a housing 327 (optionally, a cylindrical housing, as shown for example in FIG. 3A) which accommodates one or more motors (as shown for example in FIG. 4), for example, a motor 329 for driving linear movement of the guidewire and a motor 331 for driving roll movement of the guidewire. In some embodiments, the one or more motors actuate a driving assembly 333 which comprises, for example, a plurality of opposing wheels 335, where the guidewire extends through a path defined by the plurality of wheel pairs, in between opposing wheels. In some embodiments, when the wheels rotate (for example when actuated by the linear movement motor 329), the guidewire is moved linearly (e.g. advanced or retracted). In some embodiments, when the driving assembly 333 rotates about the driving assembly long axis (for example when actuated by the roll movement motor 331), the guidewire held in between the wheels is caused to roll about its long axis.

Moving on to the base 305, in some embodiments, actuation portion 309 includes one or more motors (shown for example in FIG. 4), for example, a motor 337 which drives linear movement of the microcatheter, and a motor 339 which drives roll of the guiding catheter. In some embodiments, motor 337 actuates a driving assembly 341 which comprises a plurality of opposing wheels 343 which when rotated move the microcatheter linearly. In some embodiments, motor 339 actuates rotation of a gear 342 which is part of a guiding catheter adaptor 351 (seen best in FIGS. 5, 6A), so that rotation of the gear rolls the guiding catheter about the guiding catheter long axis. (In some embodiments, linear movement of the guiding catheter is by sliding movement of the base relative to the platform on which it is mounted).

In some embodiments, the alignment stage 307 of the base defines a designated recess 345 (see for example FIG. 5, 6A) for example formed as an elongate slot extending along at least a portion of the stage length, for receipt of the microcatheter. In some embodiments, another recess 347 is defined as a linear extension of recess 345, where recess 347 is shaped and sized to receive the guiding catheter Y-connector 344.

In use, according to some embodiments, a user inserts the pre-prepared telescopic arrangement of tools such that the microcatheter is placed inside recess 345, and the guiding catheter Y-connector 344 is placed inside recess 347. In some embodiments, a more proximal portion of the microcatheter extends between the barrel and the base, with the microcatheter proximal end optionally being attached to the barrel via a connector 348 (where the guidewire that passes through the barrel enters the microcatheter lumen, see FIG. 4). In some embodiments, connector 348 is flexible (e.g. formed of an elastic or bendable material, such as silicon, rubber) so that optionally the connector itself is allowed to bend along with the micro catheter which curves along a path between the barrel and the base. Optionally, at least a portion of the connector is formed of a transparent material (for example so that substances injected through the connector can be visible).

In some embodiments, a topography of the alignment stage is shaped to direct the one or more tools into their designated recesses. In some embodiments, the alignment and/or directing of tools into their recesses and optionally to a selected (e.g. initial) rotational orientation of the tool is facilitated by one or more protrusions and/or recesses of the alignment stage. For example, in some embodiments, for setting an initial rotational orientation of the guiding catheter, adaptor 351 of the guiding catheter (see FIG. 5, FIG. 6A), which includes for example a Y-connector 344 or a T-connector and optionally one or more gears such as gear 342, is received within a designated recess 353 (seen best in FIG. 5) of the alignment stage.

In some embodiments, a branch 354 of the connector extends at an angle to the tool (or tool arrangement) long axis, so that positioning of the branch inside its designated recess sets a position of the tool along a long axis of the alignment stage and/or along a width axis of the alignment stage. Optionally, the branch is perpendicular to the tool long axis.

In some embodiments, insertion of at least a portion of adaptor 351, such as Y-connector 344 into recess 353 secures the guiding catheter Y-connector 344 in recess 347, reducing or preventing unintentional movement (e.g. pull out) of the guiding catheter. Optionally, insertion of the Y-connector or a portion of it (e.g. branch 354) into the recess centers the tool with respect to the alignment stage.

Additionally or alternatively to alignment of a tool with the aid of a connector of the tool, in some embodiments, one or more stabilizing elements such as rods or protrusions (not shown) are optionally placed (e.g. press-fitted, threaded) onto the arrangement of tools prior to placing of the arrangement on the alignment stage, and those stabilizing elements are then received in designated recesses of the alignment stage.

In some embodiments, the alignment stage defines a recess 359 (for example comprising a partial disc shape, see FIGS. 6A-B) in which gear 342 of the guiding catheter adaptor 351 is seated.

In some embodiments, one or more securing elements such as bridge 355 (see FIG. 5) are placed to hold the tools (e.g. the microcatheter) inside the recess, for example preventing the tool from being unintentionally pulled out of the recess.

Then, in some embodiments, the user positions the actuation portion 309 (see FIGS. 3A, 4) of the base on top of the alignment stage (for example by folding or otherwise placing the actuation portion over the alignment stage), such that driving assembly 341 (see FIG. 4) is aligned relative to recess 345 (see FIG. 5). In some embodiments, closing of the actuation portion over the alignment stage positions wheels 343 (FIG. 4) of the driving assembly at diametrically opposing positions across recess 345 (FIG. 5) so that the wheels contact the microcatheter received within the recess. In some embodiments, closing of the actuation portion places a gear 349 (see FIG. 4) coupled to motor 339 in interference with gear 342, such that rotation of gear 349 rotates gear 342 which thereby rolls the guiding catheter received at the alignment stage.

In some embodiments, the actuation portion is configured to interlock to the alignment stage, for example by an interlock 357 (see FIG. 3A, FIG. 4). In an example, interlock 357 comprises a snap-fit attachment. Optionally, interlock 357 is configured so that its closure releases a mechanical and/or electrical lock which only once released enables, for example, operation of the driving assemblies which move the tools.

In some embodiments, as shown for example in FIG. 3A, the device comprises light indicators 361 (e.g. a series of LED lights) located for example on an external side of a housing of the one or more units. In some embodiments, the light indicators are configured to indicate one or more of: receipt of a tool in its unit, a direction of linear movement of the tool, a speed of movement of the tool, unit malfunction, tool condition (e.g. buckling or bending).

### Changing a Spatial Configuration of the Robotic Device

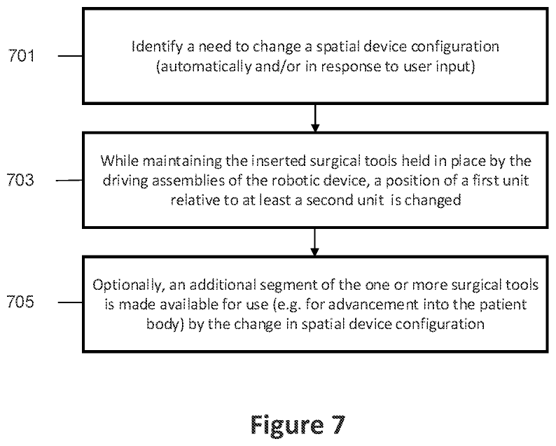

FIG. 7 is a flowchart of a method for changing a spatial configuration of the robotic device, according to some embodiments.

In some embodiments, a need for changing a spatial configuration of the device is identified (701). Reasons for changing a spatial configuration of the device may include: a need to provide or modify visual access and/or physical access for imaging devices and/or other instruments used in the procedures; a need to effectively lengthen a usable length of an elongate surgical tool manipulated by the device, as further described herein; a need to reposition connector(s) of the elongate surgical tool, for example to relocate a connector of a tool relative to the device and/or the patient such as to facilitate injection of materials through the connector;

In some embodiments, identifying is performed automatically by the device, for example in response to an indication obtained by one or more sensors (such as an indication that additional usable tool length is required); in response to signals received from one or more encoders of the system, for example in response to a count of motor rotations based on which a decision for releasing additional tool length is received, and/or other system indications. Additionally or alternatively, identifying is by a user of the device (for example by visually detecting and/or tactile sensing that additional useable tool length is needed).

In some embodiments, while the surgical tools are held in place inside the device units, for example, grasped by the wheels of the driving assemblies in each of the units, a position of a first unit relative to at least a second unit of the device is changed (703). In some embodiments, the change in position is by adjustment of a coupler (e.g. a frame) which couples the units to each other.

In some embodiments, the change in position includes moving the first unit from a position in which the first unit is held above the second unit (vertically oriented relative to the second unit) to a position in which the first unit is held adjacent the second unit (horizontally oriented relative to the second unit) or at an angle to the second unit (inclined setup).

As referred to herein, a position of a unit may include a position of the housing of the unit and/or refer to an orientation of a long axis defined by the driving assembly of the unit which contacts and optionally holds the tool. For example, in a vertical orientation of the units, a driving assembly of the first unit may be arranged on a plane parallel to that of a driving assembly of the second unit; in a horizontal orientation of the units, a driving assembly of the first unit may be arranged linearly to the driving assembly of the second unit (e.g. axially behind the driving assembly of the second unit); in an angular orientation of the units, a driving assembly of the first unit may extend at an angle (e.g. at an angle between 1-179 degrees) relative to the driving assembly of the second unit.

Optionally, in some embodiments, the change in spatial configuration makes an additional segment of a surgical tool (e.g. a guidewire and/or a microcatheter) manipulated by the device available for use by the device (705). For example, by placing the first unit linearly behind the second unit or at an angle smaller than 90 degrees relative to the second unit, instead of directly above the second unit, additional microcatheter and/or guidewire tool segments may be made available for use due to that a curved segment that passed between the first and second units is now “shortened” and optionally the curve is avoided. Once additional tool length is made available for use, the tool can be advanced further, for example further into the patient body.

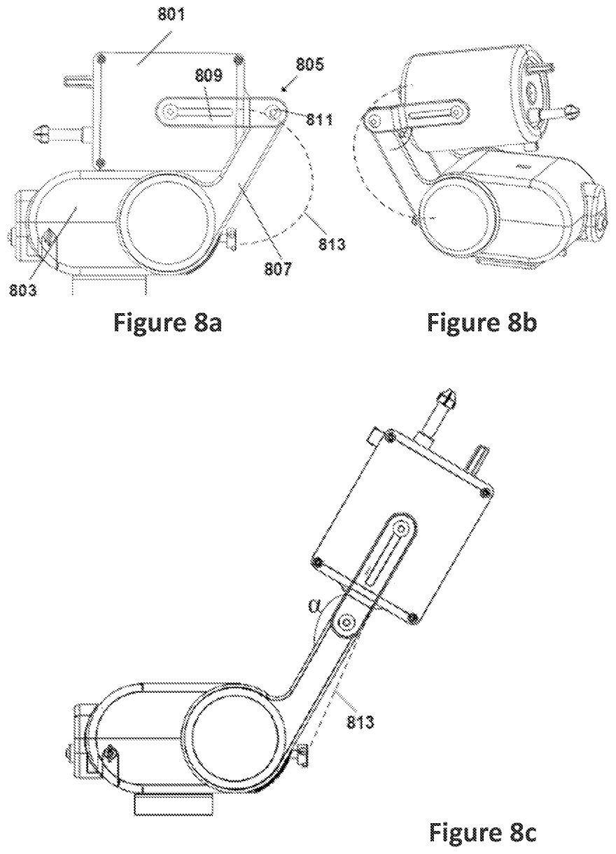

FIGS. 8A-C are side views of an exemplary embodiment of the robotic device in a vertical setup and in an inclined setup, according to some embodiments.

In some embodiments, as shown for example in FIG. 8A, the barrel unit 801 is vertically aligned with respect to the base unit 803, for example so that long axes of driving assemblies (not shown) of the surgical tools received and manipulated by the units are parallel to each other. In some embodiments, a coupler 805 holds the two units relative to each other. In some embodiments, coupler 805 comprises a vertical rail 807 and a lever portion 809, coupled to each other at a joint 811, for example, a hinge or pivot joint. In some embodiments, in the vertical alignment, a segment of a tool 813 (such as a microcatheter), that extends from one unit to the other curves along a path between the units. Potential advantages of a vertical orientation of the units may include: providing an available space (e.g. a volume external to the units) for changing the curve of the tool, for example, lengthening or shortening the tool segment that curves, optionally based on use of the tool inside the patient; minimizing a spatial interference of the device as a whole.

In some embodiments, as shown in the examples of FIGS. 8B-C, the barrel unit 801 is moved and held at an inclined position relative to the base unit 803. In some embodiments, coupler 805 is adjusted by changing an angle α of the lever portion 809 relative to the vertical rail, for example from a sharp or perpendicular angle at the vertical alignment of the units to an obtuse angle (e.g. between 90-180 degrees) in the inclined setup.

In some embodiments, the relative orientation of the units is maintained by a biasing component. In an example, the coupler 805 comprises a spring (e.g. at the lever portion, not shown) and once the spring is released, the lever portion is elastically bounced by the spring, changing the orientation of the units from a vertical orientation to an inclined orientation.

In some embodiments, the change in spatial configuration shortens the length of the segment of the tool 813 that extends between the units, for example by the change from a curved path (for example as shown in FIG. 8A) to a substantially linear path (for example as shown in FIG. 8C). In an example, the length of the segment passing between the units (externally to the housings of the units) is shortened by the change of spatial device configuration by at least 20%, 40%, 70% or intermediate, larger or smaller amount.

In some embodiments, a minimal length of the segment extending between the units is dictated by a rigid proximal portion of the tool (e.g. the microcatheter) where for example a connector and/or a luer are configured, and/or where a proximal stiffer end catheter is provided.

In some embodiments, use of a soft and/or flexible connector may be less restrictive when changing the curved segment length and/or otherwise changing a pathway along which the tool extends externally to the unit housings. In an example, in the vertical configuration of the units in which the tool curves between the units, a soft and/or flexible connector may allow for a shorter tool segment to extend between the units.

In some embodiments, the change in spatial configuration is controlled remotely, for example via a remote controller of the system.

Detection and Control of Bending and/or Buckling of an Elongate Surgical Tool

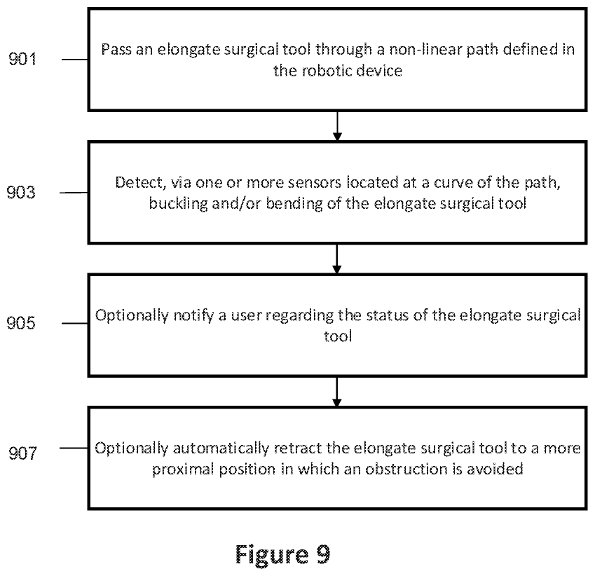

FIG. 9 is a flowchart of a method for detection and/or control of buckling and/or bending of an elongate surgical tool at least partially received within the robotic device, according to some embodiments.

In some embodiments, the robotic device defines at least one non-linear (i.e. curved) path for an elongate surgical tool to extend through (901). In some embodiments, the path is mostly linear (e.g. linear along 60%, 80%, 95% or intermediate, longer or shorter percentage of its length) yet comprises an axially-offset position that extends away from the long axis of the path. In some embodiments, the axially offset position includes a curved portion.

In some embodiments, the curved portion forces the tool to bend when passing through the curvature, for example so that if/when a distal end of the tool hits an obstruction (e.g. by encountering tissue or other structure inside the patient body), a direction and/or axial location of buckling of the tool (along the tool length) will be set by the curved path. In some embodiments, the tool segment that extends through the curved path rests freely inside the curve, for example, is not restricted by any structural elements so that the segment can be free to bend, especially when the tool distal end encounters an obstruction.

In some embodiments, one or more sensors located at or adjacent the curved path are configured to detect buckling and/or bending of the tool for example by sensing force (e.g. pressure) applied by the tool onto one or more sensors located at the curvature of the path defined in the robotic device (903).

In some embodiments, upon detection of force (e.g. pressure) applied onto the one or more sensors by the tool, a user is notified regarding a condition of the tool (905), for example notified that the tool is encountering resistance at its distal end. Such notification may be provided via a visible and/or audible and/or tactile indication on the robotic device and/or on its remote controller (for example, a vibration of the remote controller). In some embodiments, the system controller is configured to generate notifications regarding the tool condition, such as buckling or bending.

In some embodiments, optionally, the tool is automatically retracted (e.g. by actuation of the driving assembly to retract the tool linearly backwards) for example to a more proximal position in which an obstruction at the tool distal end is avoided (907).

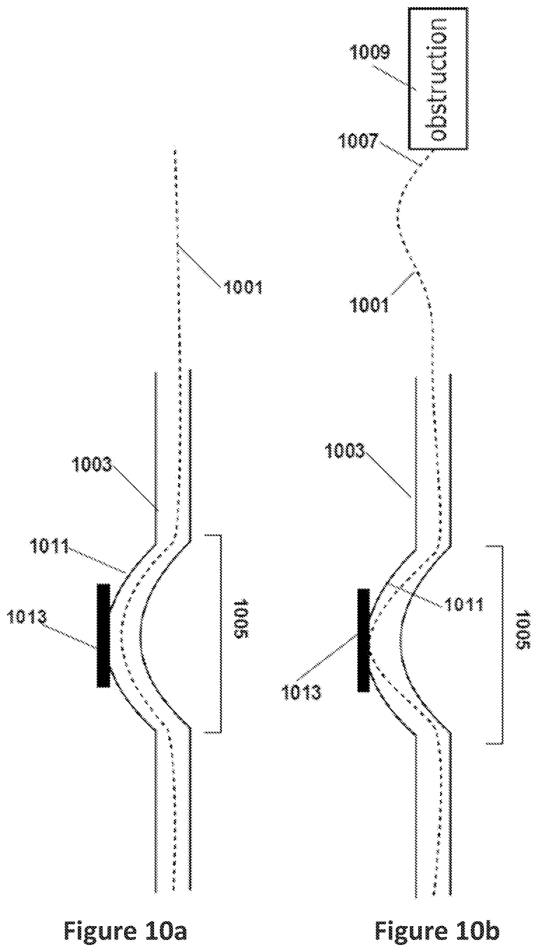

FIGS. 10A-B schematically illustrate a non-linear path for an elongate surgical tool where sensing of buckling and/or bending of the tool is performed, according to some embodiments.

In some embodiments, tool 1001 is received within a path 1003 of the robotic device which comprises one or more curved sections 1005. In some embodiments, the tool rests freely at least in the curved section and is not restrained.

In some embodiments, in use, as shown for example in FIG. 10B, if tool 1001 encounters an obstruction 1009, for example at a distal end 1007 of the tool, the curved section sets a direction of bending of the tool as a more proximal portion of the tool which extends within the curved section is forced against an inner wall 1011 of the path.

In some embodiments, the curved section includes one or more sensors 1013, for example positioned at the inner wall of the path, which sense contact and/or force (e.g. pressure) applied by the tool when the tool is pushed against the wall as a result of the bend.

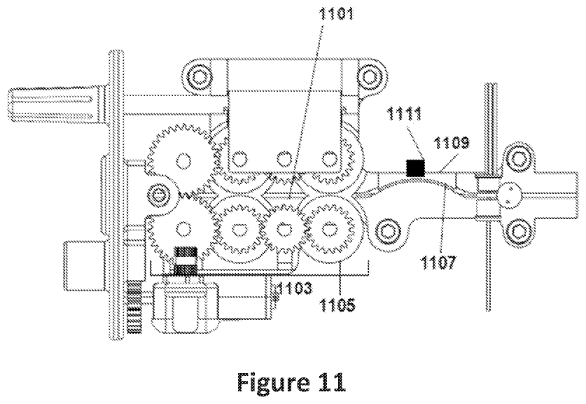

FIG. 11 is a cross section of an exemplary portion of the robotic device which includes a non-linear path for an elongate surgical tool, according to some embodiments, and as shown and exemplified in FIGS. 10A-B.

In some embodiments, a path 1101 which is defined within a unit of the robotic device (for example, the barrel unit of the device, as shown and exemplified in FIG. 3A) extends through a driving assembly 1103, for example within a space defined by the one or more wheels 1105 of the driving assembly, and continues to form a curved section 1107, optionally distally to the driving assembly. In some embodiments, the curved section is defined by walls 1109 inside the unit, for example by a curvature in the wall, a bump or protrusion or the like. In some embodiments, one or more sensors 1111 (e.g. pressures sensors, proximity sensors, contact sensors) are located at or adjacent the curved section to sense if a tool (e.g. a guidewire, in the example of barrel unit) has been pushed towards or against the wall of the path at the curve.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A robotic device for manipulation of an arrangement of a plurality of elongate surgical tools, comprising:
a first motive unit and a second motive unit, each motive unit configured to receive at least one elongate surgical tool of said arrangement, wherein an elongate surgical tool received by said first motive unit is different from an elongate surgical tool received by said second motive unit; each motive unit comprising a driving assembly and one or more motors configured for actuation of said driving assembly to move said at least one elongate surgical tool; and
a coupler attached to said first motive unit and to said second motive unit, said coupler comprising two or more portions connected to each other; said coupler configured so that movement of said two or more portions relative to each other allows movement of at least one of said first and second motive units relative to the other motive unit, while in each of said first and second motive units said elongate surgical tool remains in operable engagement with said driving assembly.

2. The robotic device according to claim 1, wherein at least said first motive unit is attached to said coupler by a reversible attachment which provides for replacing at least said first motive unit as a whole; and
wherein said coupler is configured to hold said first motive unit relative to said second motive unit at more than one spatial configuration.

3. (canceled)

4. The robotic device according to claim 2, wherein said more than one spatial configuration includes a vertical arrangement of said first motive unit relative to said second motive unit, such that long axes of said driving assemblies of said units are parallel to each other; and an inclined arrangement in which a long axis of said driving assembly of said first motive unit extends at an angle relative to a long axis of said driving assembly of said second motive unit.

5. The robotic device according to claim 1, wherein in each of said first and second motive units, no barrier exists between said driving assembly and said elongate surgical tool received within said motive unit.

6. The robotic device according to claim 1, wherein in each of said motive units, no barrier exists between said driving assembly and said one or more motors.

7. The robotic device according to claim 1, wherein one of said at least two portions of said coupler is attached to said first motive unit, and the other of said at least two portions of said coupler is attached to said second motive unit.

8. The robotic device according to claim 1, wherein movement of said two or more portions of said coupler is spring actuated.

9. The robotic device according to claim 1, wherein said elongate surgical tool received within said first motive unit comprises a guidewire, and said elongate surgical tool received within said second motive unit comprises a microcatheter, said arrangement comprising said guidewire at least partially inserted into a lumen of said microcatheter;
wherein said driving assembly of said first motive unit is positioned to contact said guidewire when said guidewire is received within said first motive unit and to one or both of move said guidewire linearly and roll said guidewire; and wherein said driving assembly of said second motive unit is positioned to contact said micro catheter when said microcatheter is received within said second motive unit and to move said microcatheter linearly; and
wherein said second motive unit comprises an attachment to a guiding catheter, said microcatheter and guidewire arrangement being at least partially inserted into a lumen of said guiding catheter.

10-11. (canceled)

12. The robotic device according to claim 9, wherein said second motive unit is slidably mounted on a platform, and wherein sliding movement of said second motive unit relative to said platform linearly moves said attached guiding catheter along with said inserted microcatheter and guidewire.

13. The robotic device according to claim 1, wherein said first motive unit is shaped as a barrel, and said coupler holds said first motive unit vertically above said second motive unit.

14. The robotic device according to claim 1, wherein said two or more portions of said coupler comprise a lever portion and a vertical rail which are connected to each other at a joint.

15-31. (canceled)

32. A method of effectively increasing a useable length of an elongate surgical tool manipulated by a said robotic device according to claim 1, said elongate surgical tool extending from an attachment with said first unit, between said first and second units, and then through a designated path of said second unit, the method comprising:
identifying a need to increase a useable length of the elongate surgical tool;
while maintaining the elongate surgical tool in attachment with said first unit and inside said designated path of said second unit, approximating an attachment of said elongate surgical tool to said first unit to an entry point of said elongate surgical tool into said designated path of said second tool, such that an additional segment of said elongate surgical tool is made available for use; and
advancing said elongate surgical tool through said designated path and further into a patient body.

33. (canceled)

34. The method according to claim 32, wherein said approximating comprises changing a spatial configuration of said first unit relative to said second unit via a coupler connected to said first and second units.

35. The method according to claim 34, wherein said changing comprises moving said first unit from a vertical position relative to said second unit to an inclined or horizontal position relative to said second unit; and wherein said moving is spring actuated.

36. (canceled)

37. The method according to claim 32, comprising controlling said approximating remotely using a remote-controller.

38. The method according to claim 32, wherein said identifying comprises one or more of: identifying in response to a signal received from a controller of said robotic device, identifying in response to user input, identifying in response to an indication obtained by one or more sensors of said robotic device.

39-47. (canceled)

48. A motive unit for manipulation of a guidewire, comprising:
a housing including:
a guidewire pre-loaded onto a designated path inside said housing;
one or more motors;
a driving assembly configured to move said guidewire, said driving assembly actuated by said one or more motors;
wherein said housing defines an external attachment to a coupler shaped and configured to hold said motive unit relative to at least a second motive unit.

49. The motive unit according to claim 48, wherein said driving assembly comprises a plurality of wheel pairs positioned in contact with said guidewire, wherein rotation of said wheels moves said guidewire linearly and wherein rotation of said driving assembly as a single unit rolls said guidewire as said guidewire is held between opposing wheels of said plurality of wheel pairs.

50. The motive unit according to claim 48, wherein said housing is shaped as a barrel and wherein a diameter of said barrel is sized according to a radius of rotation of said driving assembly as a single unit.

51-53. (canceled)

54. The motive unit according to claim 48, wherein said coupler comprising two or more rigid portions that are adjustable in position relative to each other, a first portion configured to attach to said motive unit and a second portion configured to attach to said second motive unit, wherein movement of said rigid portions relative to each other provides for selectively positioning said motive unit relative to said second motive unit.
